(19) United States
(12) Patent Application Publication
(10) Pub. No.: US 2025/0066866 A1
(43) Pub. Date: Feb. 27, 2025

(54) SUBSTITUTED DIAZENYLANILINES AS FLUORESCENCE QUENCHER AND USE THEREOF

(71) Applicant: COUNCIL OF SCIENTIFIC AND INDUSTRIAL RESEARCH AN INDIAN REGISTERED BODY INCOPORATED UNDER THE REGN., New Delhi, Delhi (IN)

(72) Inventors: Atul GOEL, Uttar Pradesh Lucknow (IN); Kundan SINGH RAWAT, Uttar Pradesh Lucknow (IN); Priyanka PANDEY, Uttar Pradesh Lucknow (IN); Ashish ARORA, Uttar Pradesh Lucknow (IN); Niti KUMAR, Uttar Pradesh Lucknow (IN); Damodara REDDY NANDARAPU, Uttar Pradesh Lucknow (IN)

(73) Assignee: COUNCIL OF SCIENTIFIC AND INDUSTRIAL RESEARCH AN INDIAN REGISTERED BODY INCOPORATED UNDER THE REGN., New Delhi, Delhi (IN)

(21) Appl. No.: 18/711,488

(22) PCT Filed: Nov. 23, 2022
(86) PCT No.: PCT/IN2022/051025

§ 371 Date: May 17, 2024

(30) Foreign Application Priority Data
Nov. 23, 2021 (IN) 202111054081

(51) Int. Cl. C12Q 1/70; C07C 245/08; C12Q 1/6818; C12Q 1/6851

(52) U.S. Cl. CPC C12Q 1/701; C07C 245/08; C12Q 1/6818; C12Q 1/6851

(57) ABSTRACT

The present invention relates to substituted-diazenylanilines of the formula I and their nucleotide conjugates, complexes, salts which may be used potentially as fluorescent quenchers in chemical and biological sciences such as cell imaging applications, diagnostics, fluorescent and non-fluorescent tags, pharmaceuticals and other useful applications, and a process of preparing said new compounds. More particularly, the present invention relates to 2,2′-((4-((2,5-disubstituted-4-((4-nitrophenyl)diazenyl)phenyl)diazenyl)-2/3-substituted-phenyl)azanediyl)dialkanol, processes for preparing said compounds and their use as fluorescent quenchers in cell imaging applications, diagnostics, fluorescent and non-fluorescent tags, pharmaceuticals and other useful applications.

### FIELD OF THE INVENTION

The present invention relates to fluorescence quenchers. The present invention particularly relates to substituted-diazenylanilines and their nucleic acid conjugates, complexes and salts which can be used potentially as fluorescent quenchers in chemical and biological sciences such as cell imaging applications, diagnostics, fluorescent and non-fluorescent tags, pharmaceuticals and other useful applications. The present invention also relates to the synthesis of substituted-diazenylanilines and their nucleic acid conjugates, complexes and salts. More particularly, the present invention relates to 2,2′-((4-((2,5-disubstituted-4-((4-nitrophenyl)diazenyl)phenyl)diazenyl)-2/3-substituted-phenyl)azanediyl)dialkanol, processes for preparing the said compounds and their uses as fluorescence quenchers in cell imaging applications, diagnostics, fluorescent and non-fluorescent tags, pharmaceuticals and other useful applications.

### BACKGROUND OF THE INVENTION

The development cost-effective techniques for detection and quantitation of chemical, and biological substances have widely enhanced high-tech innovations in the field of drugs, diagnostics and devices. It has become one of the prime areas to identify substances from micro levels to whole peptide, protein range as well as nucleic acid to other pharmaceutically important materials. These studies are intricately intertwined with our life as they play vital role in identifying diseases at the same time, they can be useful to detect and/or quantify biologically significant entity.

The methods of identifying analytes of diagnostic value could be based on certain set of their distinct characteristics to bind with chemical and biological environment. So far there are many binding methods such as antigen-antibody and protein-enzyme interactions, nucleic acid modification systems (northern blotting), and labelling techniques. A wide variety of labels have been developed for fast and efficient tabletop labelling for oligonucleotide, and these methods are useful for making small amounts of detecting probe as well as when required for mutational analysis.

Labels that are detectable using fluorescence spectroscopy are of quite interest because synthesis of fluorescent labels is tunable. We can derivatize them by simply introducing different groups to generate a wide variety of fluorescent label for different kind of moieties. Along with that they are easily commercially available. This method is based on the ability of fluorescent compounds to transfer absorbed energy from light to nearby molecules and has been utilized for the development of homogeneous methods of nucleic acid detection.

So as to achieve robust, sensitive and well specific real-time nucleic acid multiplication assays it is absolutely necessary to use proper fluorophore and quencher label pairs. This includes type of hybridization probe used in the assay, and the number of targets to be detected.

In their excited state, fluorophores may lose excitation energy by several means aside from emission of an energy photon. Such fluorescence quenching can take place by molecular motion (dynamic quenching), excited state complexation with other substances (photobleaching), contact quenching (static quenching), or energy transfer to another molecule (fluorescence resonance energy transfer, or FRET). Many nucleic acid fluorescence detection techniques use probes with fluorescent labels work by quenching of fluorescence of an adjacent second fluorescent label, or by using fluorescent-Quencher pair. The probes which are dual-labeled with reporter and quencher dyes, measure changes in their fluorescence to monitor any biochemical events. These events are responsible for change in the reporter-quencher distance, which results in observed change in fluorescence. (Chem. Commun., 2010, 46, 8154-8156)

Real-time nucleic acid amplification assays have remarkable capability to get better qualitative and quantitative results. In addition, these assays can be carried out in sealed tubes, avoiding contamination. Fluorescent nucleic acid hybridization probes contain wide range of different coordinating fluorophore and quencher pairs. Some methods are based on pair of mutually complementary oligodeoxyribonucleotides, in which one of the oligodeoxyribonucleotides remain as a probe for a single-stranded target sequence. The 5′ end of one oligodeoxyribonucleotide is labeled with a donor fluorophore and the 3′ end of the other oligodeoxyribonucleotide is labeled with an acceptor fluorophore. (Nucleic Acids Research, 2002, Vol. 30 No. 21 e122).

One of the important applications for probes including a reporter-quencher molecule pair is their use in nucleic acid amplification reactions, such as polymerase chain reactions (PCR), to detect the presence and amplification of a target nucleic acid sequences. (Acc. Chem. Res. 2011, 44, 2, 83-90).

In the TaqMan assay, the donor and quencher are preferably located on the 3′- and 5′-ends of the probe, for the assay as the efficiency of energy transfer decreases with the inverse sixth power of the distance between the reporter and quencher. Thus, if the quencher is not close enough to the reporter to achieve the most efficient quenching the background emissions from the probe can be quite high. (Nucleic Acids Res. 2011, 39, e112).

Fluorescent-Quencher linear pair probes are the standard tool for real-time PCR, intense signal to noise ratio, low cost, and compatibility with different PCR techniques have made them perfectly suitable as industrial marker standard for gene quantification in a wide range of applications. Black Hole Quencher dyes BHQ0, BHQ1, BHQ2, and BHQ3 have been used to quench across the entire visible spectrum range. FAM and BHQ dyes show top-rated performance, as reviewed in different scientific reports. TaqMan probes are used for quantitative real-time PCR analysis of gene expression, which includes PCR primers and a TaqMan probe with a dye label (FAM) on the 5′ end and a minor groove binder (MGB) and non-fluorescent quencher (NFQ)/Dark quencher on the 3′ end. BHQ-1 is used to quench green and yellow dyes, such as FAM, TET, and HEX. BHQ-2 and BHQ-3 are reported for quenching orange or red dyes, such as TAMRA, Texas Red, and Cy 5.

### Objects of the Invention

The main object of the present invention is to provide substituted-diazenylanilines and their nucleic acid conjugates, complexes and salts which can be used potentially as fluorescent quenchers in chemical and biological sciences such as cell imaging applications, diagnostics, fluorescent and non-fluorescent tags, pharmaceuticals and other useful applications.

Another object of the present invention is to provide a process for preparing the substituted-diazenylanilines and their nucleic acid conjugates, complexes and salts.

Yet another object of the present invention is to use the substituted-diazenylanilines and their nucleic acid conjugates, complexes and salts in chemical and biological sciences such as cell imaging applications, diagnostics, fluorescent and non-fluorescent tags, pharmaceuticals and other useful applications

### SUMMARY OF THE INVENTION

Accordingly, the present invention relates to substituted-diazenylanilines, their synthesis and study of fluorescence quenching properties and their nucleic acid conjugates, complexes, salts which can be used potentially as fluorescent quenchers in chemical and biological sciences such as cell imaging applications, diagnostics, fluorescent and non-fluorescent tags, pharmaceuticals and other useful applications, and a process of preparing said new compounds.

Accordingly, the present invention provides a compound of general formula I, nucleic acid conjugates, complexes and salts thereof,

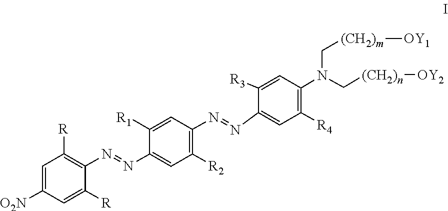

wherein R is independently selected from the group consisting of hydrogen and halogen;
R₁ and R₂ are independently selected from the group consisting of hydrogen, substituted or unsubstituted (C₁-C₄) alkyl and (C₁-C₆) alkoxy;
R₃ is selected from the group consisting of hydroxy, halogen, (C₁-C₆) alkoxy, substituted or unsubstituted (C₁-C₄) alkyl, thioalkyl (S C₁-C₆), methylamino and dimethylamino;
R₄ is selected from the group consisting of hydrogen, hydroxy, halogen, (C₁-C₆) alkoxy, and substituted or unsubstituted (C₁-C₄) alkyl;
m and n are selected from 0 to 3;
Y₁ and Y₂ are independently selected from the group consisting of hydrogen, (C₁-C₆) alkyl, glycol, substituted or unsubstituted alkylaryl, oxo-alkanoic acid, epoxy, N-hydroxysuccinimide ester, N-hydroxybenztriazole ester, acid halide, acyl imidazole, thioester, p-nitrophenyl ester, alkyl ester, mononucleotide unit, two or more mononucleotide units with or without separate phosphate or polyphosphate groups linked by nucleoside groups.

In a preferred embodiment of the present invention the compound of formula I is selected from the group consisting of:
i. 2,2′-((4-((2,5-dimethoxy-4-((4-nitrophenyl)diazenyl)phenyl)diazenyl)-3-methoxyphenyl)azanediyl)bis(ethan-1-ol) (1),
ii. 2,2′-((4-((2,5-dimethoxy-4-((4-nitrophenyl)diazenyl)phenyl)diazenyl)-3-ethoxyphenyl)azanediyl)bis(ethan-1-ol) (2),
iii. 2,2′-((4-((2,5-dimethoxy-4-((4-nitrophenyl)diazenyl)phenyl)diazenyl)-3-propoxyphenyl)azanediyl)bis(ethan-1-ol) (3),
iv. 2,2′-((3-butoxy-4-((2,5-dimethoxy-4-((4-nitrophenyl)diazenyl)phenyl)diazenyl)phenyl)azanediyl)bis(ethan-1-ol) (4),
v. 2,2′-((4-((2,5-dimethoxy-4-((4-nitrophenyl)diazenyl)phenyl)diazenyl)-3-(hexyloxy)phenyl)azanediyl)bis(ethan-1-ol) (5),
vi. 2,2′-((4-((2,5-dimethoxy-4-((4-nitrophenyl)diazenyl)phenyl)diazenyl)-3-methylphenyl)azanediyl)bis(ethan-1-ol) (6),
vii. 2,2′-((4-((2,5-dimethoxy-4-((4-nitrophenyl)diazenyl)phenyl)diazenyl)-2,5-dimethoxyphenyl)azanediyl)bis(ethan-1-ol)(7),
viii. 2,2′-((3-bromo-4-((2,5-dimethoxy-4-((4-nitrophenyl)diazenyl)phenyl)diazenyl)phenyl) azanediyl)bis(ethan-1-ol)(8),
ix. 2,2′-((4-((4-((2,6-dichloro-4-nitrophenyl)diazenyl)-2,5-dimethoxyphenyl)diazenyl)-3-methoxyphenyl)azanediyl)bis(ethan-1-ol) (9),
x. 2,2′-((4-((2,5-dimethoxy-4-((4-nitrophenyl)diazenyl)phenyl)diazenyl)-3-hydroxyphenyl)azanediyl)bis(ethan-1-ol) (10),
xi. 4-(2-((2-(bis(4-methoxyphenyl)(phenyl)methoxy)ethyl)(4-((2,5-dimethoxy-4-((4-nitrophenyl)diazenyl)phenyl)diazenyl)-3-methoxyphenyl)amino)ethoxy)-4-oxobutanoic acid (11),
xii. 4-(2-((2-(bis(4-methoxyphenyl)(phenyl)methoxy)ethyl)(4-((2,5-dimethoxy-4-((4-nitrophenyl)diazenyl)phenyl)diazenyl)-3-ethoxyphenyl)amino)ethoxy)-4-oxobutanoic acid (12),
xiii. 4-(2-((2-(bis(4-methoxyphenyl)(phenyl)methoxy)ethyl)(4-((2,5-dimethoxy-4-((4-nitrophenyl)diazenyl)phenyl)diazenyl)-3-propoxyphenyl)amino)ethoxy)-4-oxobutanoic acid (13),
xiv. 4-(2-((2-(bis(4-methoxyphenyl)(phenyl)methoxy)ethyl)(3-butoxy-4-((2,5-dimethoxy-4-((4-nitrophenyl)diazenyl)phenyl)diazenyl)phenyl)amino)-ethoxy)-4-oxobutanoic acid (14),
xv. 4-(2-((2-(bis(4-methoxyphenyl)(phenyl)methoxy)ethyl)(4-((2,5-dimethoxy-4-((4-nitrophenyl)diazenyl)phenyl)diazenyl)-3-(hexyloxy)phenyl)amino)-ethoxy)-4-oxobutanoic acid, (15) and
xvi. 4-(2-((2-(bis(4-methoxyphenyl)(phenyl)methoxy)ethyl)(4-((2,5-dimethoxy-4-((4-nitrophenyl)diazenyl)phenyl)diazenyl)-3-methylphenyl)amino)-ethoxy)-4-oxobutanoic acid (16).

The present invention also provides a process for the preparation of compound of general formula I and nucleic acid conjugates, complexes, salts thereof, wherein R, R₁, R₂ R₃, R₄, m, n, Y₁ and Y₂ are as defined above, comprising the steps of:

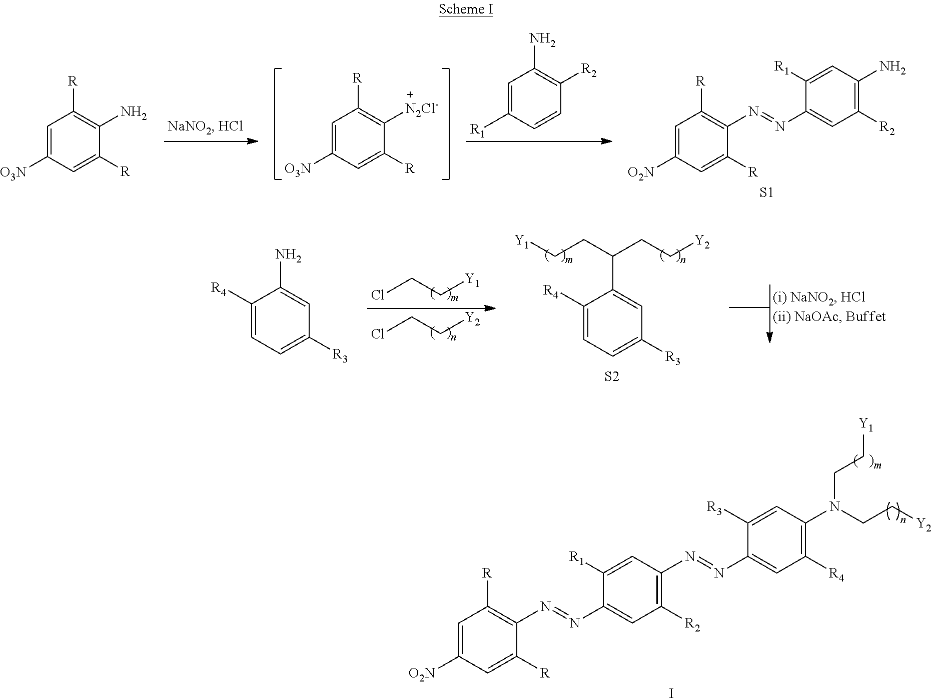

a. reacting a solution of unsubstituted or substituted 4-nitroaniline in HCl with a solution of sodium nitrite in distilled water at 0° C. to form diazonium salts followed by reacting with substituted aniline to form a compound of formula S1;
b. reacting a mixture of substituted aniline with substituted alkylhalide in the presence of base to form a compound of formula S2;
c. reacting a compound of formula S1 in HCl with a solution of sodium nitrite to form a diazonium salt, which was then reacted with a compound of formula S-2 in the presence of NaOAc buffer to obtain a compound having general formula I and
d. isolating the compound of general formula I from the reaction mixture and purifying by washing with organic solvents or by chromatography.

In a preferred embodiment of the present invention step a-c of the above mentioned process is carried out in the presence of an organic solvent selected from CH₃CN, Dimethylsulphoxide, water and tetrahydrofuran at a temperature ranging between 0° C. to 100° C. for a period ranging between 1 minute to 3 days. In a preferred embodiment the present invention provides a process comprises the steps of:

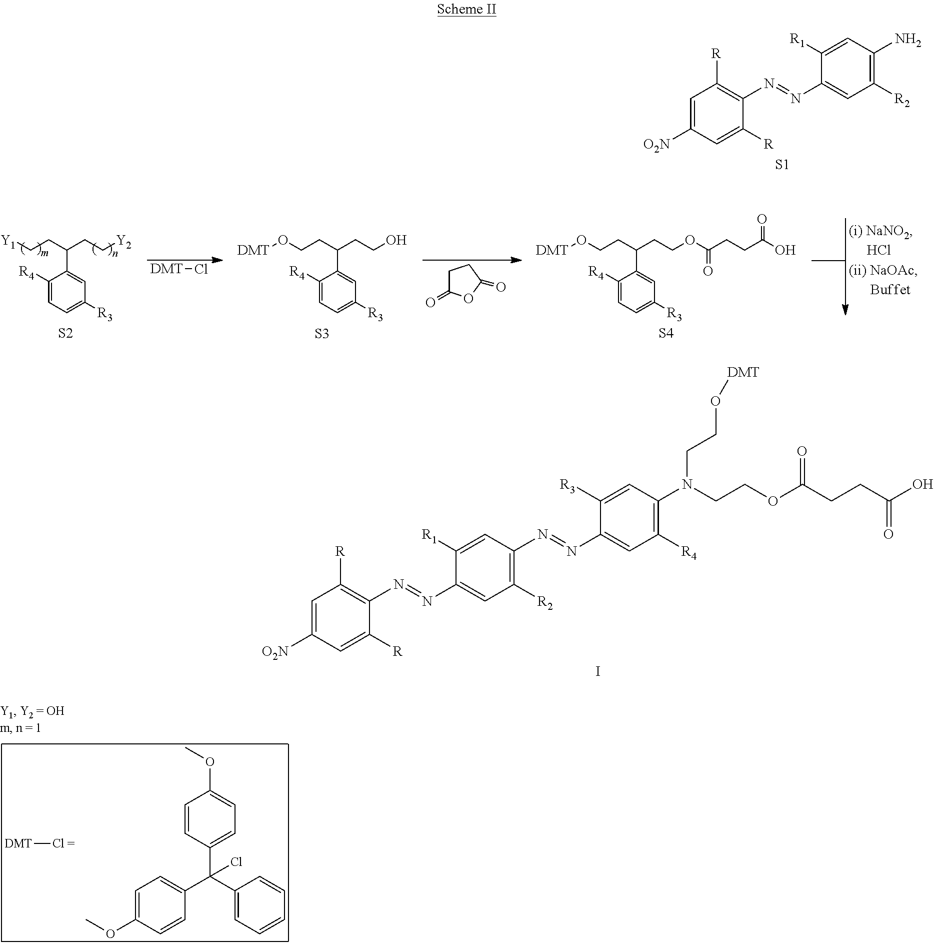

a) reacting compound S2, wherein Y₁, Y₂=OH and m,n=1 in dry DCM in the presence of a base (DIPEA) with DMT-Cl at room temperature under inert atmosphere to afford compound S3;
b) reacting S3 with succinic anhydride, and DMAP in organic solvent for 12-48 h to afford compound S4 and
c) reacting the diazonium salt of S1 with S4 to obtain the compound having general formula I.

The present invention also provides a process for the preparation of conjugate compound of general formula I wherein Q is the compound of formula 1 comprising the steps of:

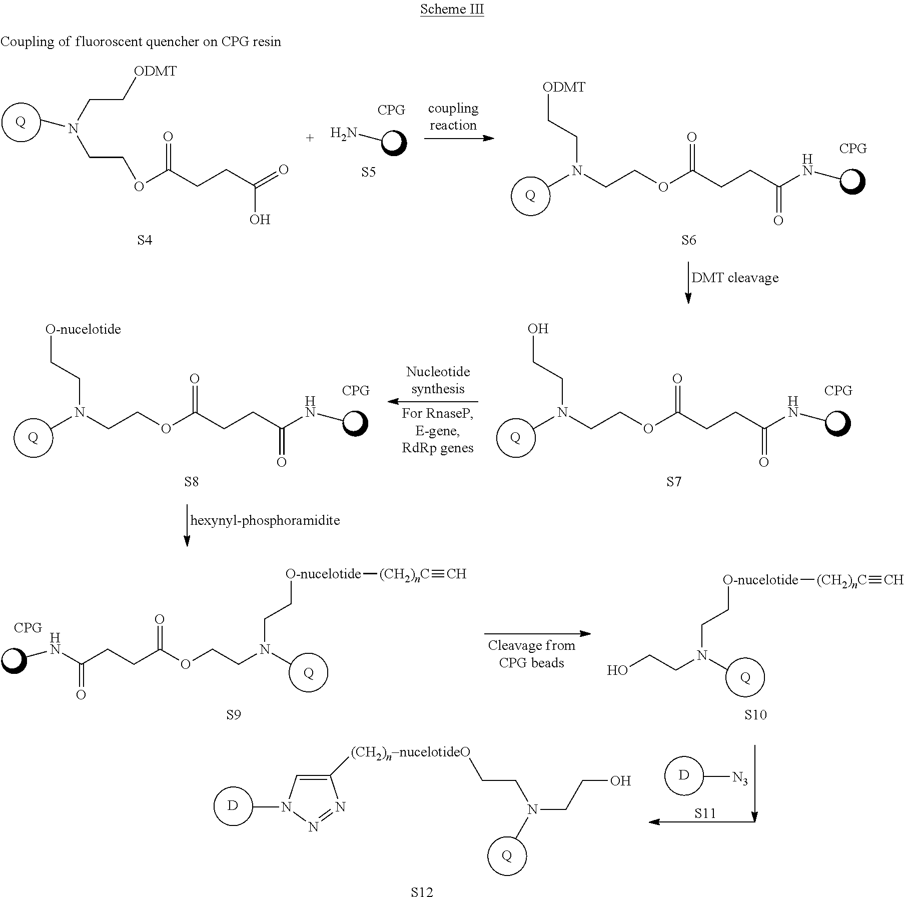

a. coupling of a compound of formula S4 with the amine functionality of the solid support CPG beads of formula S5 to produce S6 followed by deprotection of DMT group to obtain S7;
b. reacting S7 with nucleotide phosphoramidites to form oligonucleotide S8 followed by 5′-modiciation with hexynyl-phosphoramidite to afford product S9 and
c. treating S9 with a base for cleaving the oligonucleotide from the solid support to obtain S10 and
d. reacting S10 with fluorescent dye azides to furnish oligonucleotides probes of general formula S12.

The present invention provides a compound of general formula I, which is useful for analyzing nucleic acids (DNA, RNA), peptides, chemicals, pharmaceuticals, microorganisms and other biological substances of diagnostic importance.

The present invention provides a compound of general formula I, which is useful for development of diagnostic kit for detection of substances, hormones, pathogenic microorganisms and viruses, antibodies, and enzymes and nucleic acids, particularly those implicated in disease states.

The present invention provides a compound of general formula I, which is useful for the preparation of fluorescent probes, tags, markers, diagnostics, ion sensor, pharmaceuticals for detecting/trapping ions in fluorescence-based imaging and/or analysis of cells, biological fluids, chemical mixture and/or other useful applications.

The present invention also provides a composition of compound of general formula I with acetyl, azides, n-hydroxy-succinimide, oxo-alkanoic acid, glycolates, thiols, amines, hydroxides, maleimides, tetrazines, phosphate, sodium, potassium salts or phosphoramidites.

In a preferred embodiment of the present invention the compound of general formula I is useful for preparing dual labelled probe and analyzing them in single, duplexing and multiplexing in RTPCR or other related detecting systems.

In an embodiment of the invention wherein the compounds are useful for fluorescent quenchers in chemical and biological sciences.

In another embodiment of the invention wherein the compounds showing wide quenching range, in between nm 450-700 nm.

Furthermore, the present invention is to provide the compounds having the general formula I which may be used potentially as fluorescent quenchers in chemical and biological sciences such as cell imaging applications, fluorescent and non-fluorescent tags and other useful biological applications such as developing diagnostic kits.

### BRIEF DESCRIPTION OF THE DRAWINGS

The following drawing form a part of the present specification and is included to further illustrate aspects of the present disclosure. The disclosure may be better understood by reference to the drawing in combination with the detailed description of the specific embodiments presented herein:

FIG. 1 illustrates absorption spectra of newly synthesized quencher derivatives (1-9) in accordance with an embodiment of the present disclosure.

FIG. 2 illustrates comparison of absorption of BHQ-2 and CDRI-Q2 at 20 μM concentration in PCR buffer solution in accordance with an embodiment of the present disclosure.

FIG. 3 depicts fluorescence quenching of 5-FAM in the presence of different concentration of CDRI Q2 (1) in PCR buffer solution in accordance with an embodiment of the present disclosure.

FIG. 4 depicts fluorescence quenching of CY3 in the presence of different concentration of CDRI Q2 (1) in PCR buffer solution in accordance with an embodiment of the present disclosure.

FIG. 5 depicts fluorescence quenching of 5-TAMRA in the presence of different concentration of CDRI Q2 (1) in PCR buffer solution in accordance with an embodiment of the present disclosure.

FIG. 6 depicts fluorescence quenching of CalFluor in the presence of different concentration of CDRI Q2 (1) in PCR buffer solution in accordance with an embodiment of the present disclosure.

FIG. 7 depicts fluorescence quenching of Texas Red in the presence of different concentration of CDRI Q2 (1) in PCR buffer solution in accordance with an embodiment of the present disclosure.

FIG. 8 depicts fluorescence quenching of Cy5 in the presence of different concentration of CDRI Q2 (1) in PCR buffer solution in accordance with an embodiment of the present disclosure.

FIG. 9 illustrates RT-PCR data representation with the cycle threshold (Ct) on X-axis and RFU on Y-axis in accordance with an embodiment of the present disclosure.

FIG. 10 illustrates Multiplexing RT-PCR based detection of SARS-CoV-2 viral genes E and RdRp and RnaseP as housekeeping gene using positive Control; data represents with the cycle threshold (Ct) on X-axis and RFU on Y-axis, in accordance with an embodiment of the present disclosure.

FIG. 11 illustrates Multiplexing RT-PCR based detection of SARS-CoV-2 viral genes E and RdRp and Rnase P as housekeeping gene using positive RNA samples from COVID-19 positive patients; data represents with the cycle threshold (Ct) on X-axis and RFU on Y-axis, in accordance with an embodiment of the present disclosure.

### ABBREVIATIONS

PCR Polymerase Chain Reaction
TDW Triple Distilled Water

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

The invention will now be described in detail in connection with certain preferred and optional embodiments, so that various aspects thereof may be more fully understood and appreciated.

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

Accordingly, the present invention relates to the synthesis and study of fluorescence quenching properties of substituted-diazenylanilines and their nucleotide conjugates, complexes, salts which may be used potentially as fluorescent quenchers in chemical and biological sciences such as cell imaging applications, diagnostics, fluorescent and non-fluorescent tags, pharmaceuticals and other useful applications, and a process of preparing said new compounds.

The term ‘quenching probes’ refers to a quencher, which may be used to quench and/or reduce fluorescence emission in different UV-visible region to respond to a specific analyte/substance.

The present invention provides a compound of formula I:

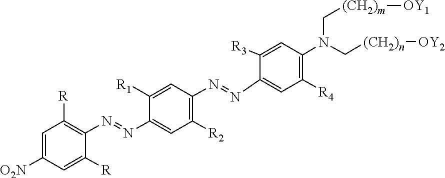

wherein
R is selected from the group consisting of hydrogen and halogen;
R₁ and R₂ are independently selected from the group consisting of hydrogen, substituted or unsubstituted (C₁-C₄) alkyl and (C₁-C₆) alkoxy;
R₃ is selected from the group consisting of hydroxy, halogen, (C₁-C₆) alkoxy, substituted or unsubstituted (C₁-C₄) alkyl, thioalkyl (S C₁-C₆), methylamino and dimethylamino;
R₄ is selected from the group consisting of hydrogen, hydroxy, halogen, (C₁-C₆) alkoxy and substituted or unsubstituted (C₁-C₄) alkyl;
m and n are numbers independently selected from 0 to 3 and
Y₁ and Y₂ are independently selected from the group consisting of hydrogen, (C₁-C₆) alkyl, glycol, substituted or unsubstituted alkylaryl, oxo-alkanoic acid, epoxy, N-hydroxysuccinimide ester, N-hydroxybenztriazole ester, acid halide, acyl imidazole, thioester, p-nitrophenyl ester, alkyl ester, phosphoramidite, mononucleotide unit, two or more mononucleotide units with or without separate phosphate or polyphosphate groups linked by nucleoside groups.

The following is a list of representative substituted-diazenylanilines compounds:
i. 2,2′-((4-((2,5-dimethoxy-4-((4-nitrophenyl)diazenyl)phenyl)diazenyl)-3-methoxyphenyl)azanediyl)bis(ethan-1-ol) (1),
ii. 2,2′-((4-((2,5-dimethoxy-4-((4-nitrophenyl)diazenyl)phenyl)diazenyl)-3-ethoxyphenyl)azanediyl)bis(ethan-1-ol) (2),
iii. 2,2′-((4-((2,5-dimethoxy-4-((4-nitrophenyl)diazenyl)phenyl)diazenyl)-3-propoxyphenyl)azanediyl)bis(ethan-1-ol) (3),
iv. 2,2′-((3-butoxy-4-((2,5-dimethoxy-4-((4-nitrophenyl)diazenyl)phenyl)diazenyl)phenyl)azanediyl)bis(ethan-1-ol) (4),
v. 2,2′-((4-((2,5-dimethoxy-4-((4-nitrophenyl)diazenyl)phenyl)diazenyl)-3-(hexyloxy)phenyl)azanediyl)bis(ethan-1-ol) (5),
vi. 2,2′-((4-((2,5-dimethoxy-4-((4-nitrophenyl)diazenyl)phenyl)diazenyl)-3-methylphenyl)azanediyl)bis(ethan-1-ol) (6),
vii. 2,2′-((4-((2,5-dimethoxy-4-((4-nitrophenyl)diazenyl)phenyl)diazenyl)-2,5-dimethoxyphenyl)azanediyl)bis(ethan-1-ol)(7),
viii. 2,2′-((3-bromo-4-((2,5-dimethoxy-4-((4-nitrophenyl)diazenyl)phenyl)diazenyl)phenyl) azanediyl)bis(ethan-1-ol)(8),
ix. 2,2′-((4-((4-((2,6-dichloro-4-nitrophenyl)diazenyl)-2,5-dimethoxyphenyl)diazenyl)-3-methoxyphenyl)azanediyl)bis(ethan-1-ol) (9),
x. 2,2′-((4-((2,5-dimethoxy-4-((4-nitrophenyl)diazenyl)phenyl)diazenyl)-3-hydroxyphenyl)azanediyl)bis(ethan-1-ol) (10),
xi. 4-(2-((2-(bis(4-methoxyphenyl)(phenyl)methoxy)ethyl)(4-((2,5-dimethoxy-4-((4-nitrophenyl)diazenyl)phenyl)diazenyl)-3-methoxyphenyl)amino)ethoxy)-4-oxobutanoic acid (11),
xii. 4-(2-((2-(bis(4-methoxyphenyl)(phenyl)methoxy)ethyl)(4-((2,5-dimethoxy-4-((4-nitrophenyl)diazenyl)phenyl)diazenyl)-3-ethoxyphenyl)amino)ethoxy)-4-oxobutanoic acid (12),
xiii. 4-(2-((2-(bis(4-methoxyphenyl)(phenyl)methoxy)ethyl)(4-((2,5-dimethoxy-4-((4-nitrophenyl)diazenyl)phenyl)diazenyl)-3-propoxyphenyl)amino)ethoxy)-4-oxobutanoic acid (13),
xiv. 4-(2-((2-(bis(4-methoxyphenyl)(phenyl)methoxy)ethyl)(3-butoxy-4-((2,5-dimethoxy-4-((4-nitrophenyl)diazenyl)phenyl)diazenyl)phenyl)amino)-ethoxy)-4-oxobutanoic acid (14),
xv. 4-(2-((2-(bis(4-methoxyphenyl)(phenyl)methoxy)ethyl)(4-((2,5-dimethoxy-4-((4-nitrophenyl)diazenyl)phenyl)diazenyl)-3-(hexyloxy)phenyl)amino)-ethoxy)-4-oxobutanoic acid, (15) and 4-(2-((2-(bis(4-methoxyphenyl)(phenyl)methoxy)ethyl)(4-((2,5-dimethoxy-4-((4-nitrophenyl)diazenyl)phenyl)diazenyl)-3-methylphenyl)amino)-ethoxy)-4-oxobutanoic acid (16).

The process for the preparation of compound of formula I wherein R, R₁, R₂ R₃, R₄, m, n, Y₁ and Y₂ are as defined above is shown in Scheme I.

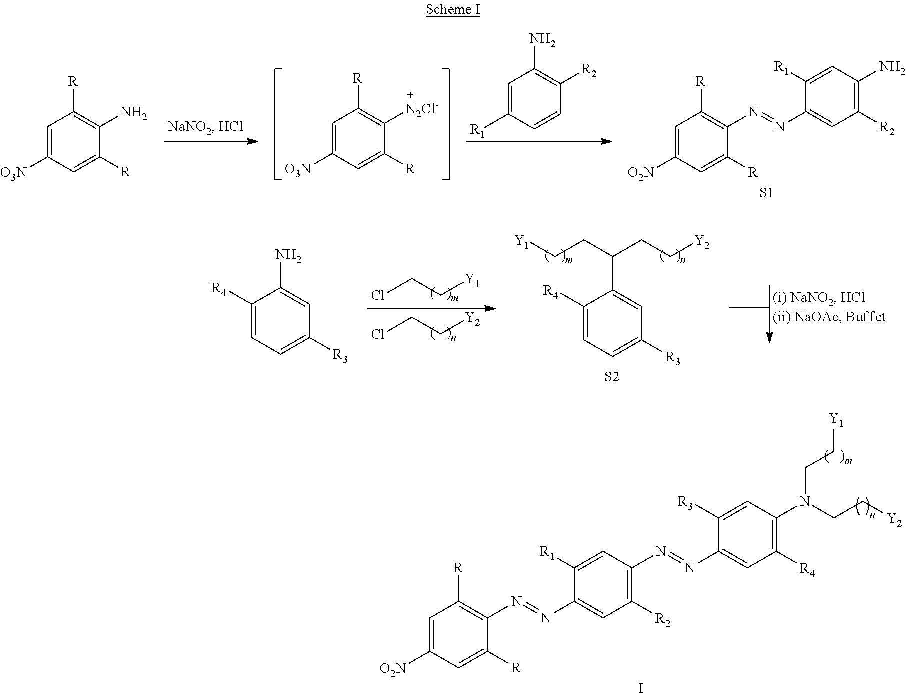

The process comprises that step of:
a. reacting a solution of unsubstituted or substituted 4-nitroaniline in HCl with a solution of sodium nitrite in distilled water at 0° C. to form diazonium salts followed by reaction with substituted aniline to form a compound having general formula S1;
b. reacting a mixture of substituted aniline with substituted alkyl halide in the presence of base to form a compound having general formula S2;
c. reacting a compound having general formula S1 in HCl with a solution of sodium nitrite to form a diazonium salt, which was then reacted with a compound having general formula S-2 in the presence of NaOAc buffer gives a compound having general formula I and
d. isolating the compound of general formula, I from the reaction mixture and purifying by washing with organic solvents or by chromatographic techniques.

The reactions are carried out in a common organic solvent particularly CH₃CN, Dimethylsulphoxide, water and tetrahydrofuran at a temperature ranging between 0° C. to 100° C. for a period ranging between 1 minute to 3 days depending upon the reactants.

In another embodiment the present invention provides a process for the preparation of preferred compounds having the formula I wherein R₁, R₂ R₃, R₄, are as defined above; Y₁, Y₂=OH and m, n=1 is shown Scheme II.

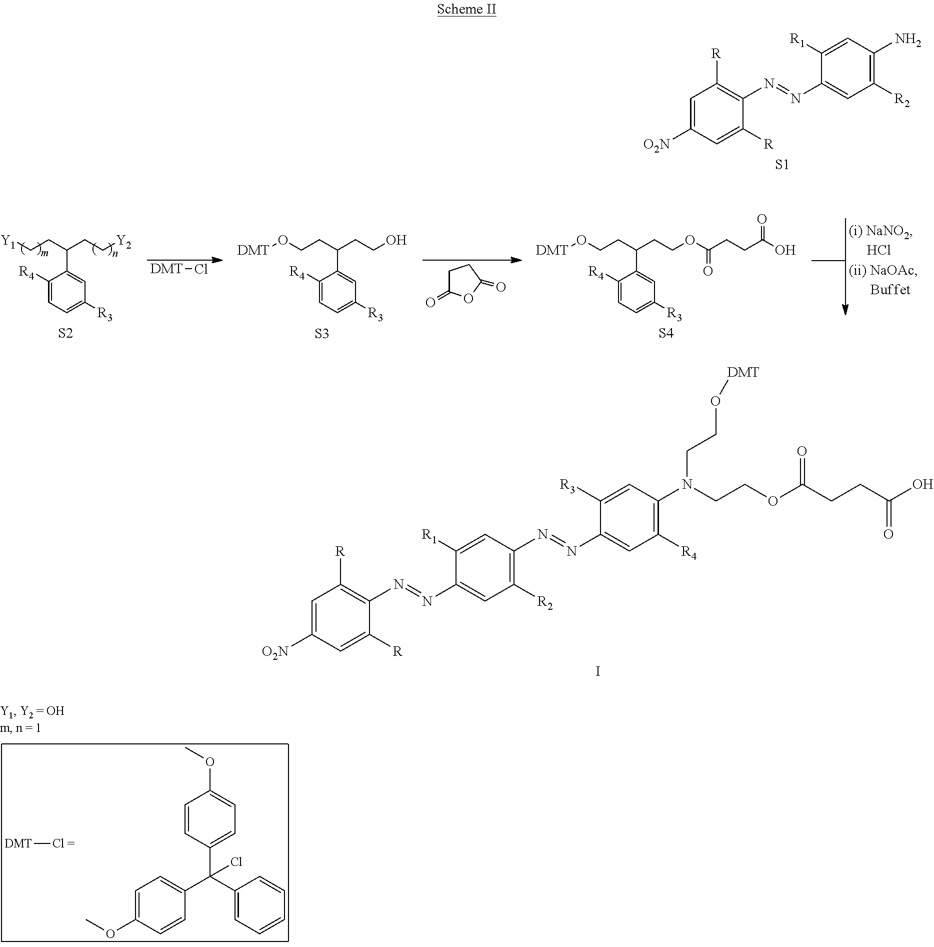

The process comprises that step of:
a) reacting a solution of unsubstituted or substituted 4-nitroaniline in HCl with a solution of sodium nitrite in distilled water at 0° C. to form diazonium salts followed by reaction with substituted aniline to form a compound having general formula S1;
b) reacting a compound having general formula S2 wherein Y₁, Y₂=OH and m, n=1 in dry DCM in the presence of a base (DIPEA) with DMT-Cl at room temperature under inert atmosphere to afford product S3;
c) reacting a compound of general formula S3 with succinic anhydride, and DMAP in organic solvent for 12-48 h to afford compound S4;
d) reacting a compound having general formula S1 in HCl with a solution of sodium nitrite to form a diazonium salt, which was then reacted with a compound having general formula S4 in the presence of a buffer (NaOAc) gives a compound having general formula I and
e) isolating the compound of general formula I from the reaction mixture and purifying by washing with organic solvents or by chromatographic techniques.

The reactions are carried out in a common organic solvent particularly CH₃CN, Dimethylsulphoxide, water and tetrahydrofuran in the presence of buffer solution at a temperature ranging between 0° C. to 100° C. for a period ranging between 1 minute to 3 days depending upon the reactants.

In an embodiment of the invention wherein the compounds are useful for fluorescent quenchers in chemical and biological sciences.

In another embodiment of the invention wherein the compounds showing wide quenching range, in between nm 450-700 nm.

Furthermore, the compounds having the general formula I can be used potentially as fluorescent quenchers in chemical and biological sciences such as cell imaging applications, fluorescent and non-fluorescent tags and other useful biological applications such as developing diagnostic kits.

### EXAMPLES

Following examples are given by way of illustration and should not construe the scope of the present invention.

### Example-1

### 2,2′-((4-((2,5-dimethoxy-4-((4-nitrophenyl)diazenyl)phenyl)diazenyl)-3-methoxyphenyl) azanediyl)bis(ethan-1-ol) (1). (CDRI-Q2)

To the stirred mixture of salt of S1 in solution of NaOAc buffer and ACN (1:1), solution of compound S2 (R3=OCH₃, R4=H) in ACN was added slowly at 0° C. After the addition, reaction mixture was stirred for 30 mins 0° C. The completion of the reaction was monitored by TLC. The reaction mixture was then filtered and washed with ACN and water (1:1) to get the product 2,2′-((4-((2,5-dimethoxy-4-((4-nitrophenyl)diazenyl)phenyl)diazenyl)phenyl)azanediyl)bis(ethan-1-ol) as purple solid. M.P.=205-206° C., MS (ESI) m/z 525 [M+H]⁺, 1H NMR (400 MHz, DMSO-d6) δ 8.47-8.40 (m, 2H), 8.09-8.03 (m, 2H), 7.62 (d, J=9.3 Hz, 1H), 7.44 (s, 1H), 7.28 (s, 1H), 6.48 (dd, J=9.4, 2.5 Hz, 1H), 6.40 (d, J=2.5 Hz, 1H), 5.03-4.80 (m, 2H), 3.99 (s, 3H), 3.97 (s, 3H), 3.94 (s, 3H), 3.68-3.57 (m, 8H). 13C NMR (101 MHz, DMSO) δ 160.61, 156.28, 154.27, 153.76, 150.62, 148.44, 147.60, 141.34, 134.36, 125.57, 123.88, 118.66, 105.80, 101.37, 100.20, 95.24, 79.64, 58.81, 56.98, 56.80, 56.49, 53.93.

### Example-2

### 2,2′-((4-((2,5-dimethoxy-4-((4-nitrophenyl)diazenyl)phenyl)diazenyl)-3-ethoxyphenyl) azanediyl)bis(ethan-1-ol) (2)

To the stirred mixture of salt of S1 in solution of NaOAc buffer and ACN (1:1), solution of compound S2 (R4=H R3=OCH₂CH₃) in ACN was added slowly at 0° C. After the addition, reaction mixture was stirred for 30 mins 0° C. The completion of the reaction was monitored by TLC. The reaction mixture was than filtered and washed with ACN and water (1:1) to get the product 2,2′-((4-((2,5-dimethoxy-4-((4-nitrophenyl)diazenyl)phenyl)diazenyl)-3-ethoxyphenyl)azanediyl)bis(ethan-1-ol) as purple solid.

M.P.=202-203° C., MS (ESI) m/z 539 [M+H]⁺, 1H NMR (400 MHz, DMSO-d6) δ 8.43 (d, J=8.6 Hz, 2H), 8.05 (d, J=8.5 Hz, 2H), 7.62 (d, J=9.3 Hz, 1H), 7.44 (s, 1H), 7.35 (s, 1H), 6.49 (d, J=9.4 Hz, 1H), 6.44-6.35 (m, 1H), 4.88 (t, J=5.1 Hz, 2H), 4.25 (q, J=7.0 Hz, 2H), 3.98 (s, 3H), 3.94 (s, 3H), 3.68-3.56 (m, 8H), 1.44 (t, J=7.0 Hz, 3H). 13C NMR (101 MHz, DMSO) δ 160.12, 156.28, 154.25, 153.70, 150.63, 148.43, 147.58, 141.26, 134.36, 125.57, 123.88, 118.51, 106.06, 101.24, 100.27, 96.67, 65.10, 58.80, 56.81, 56.67, 53.91, 15.17.

### Example-3

### 2,2′-((4-((2,5-dimethoxy-4-((4-nitrophenyl)diazenyl)phenyl)diazenyl)-3-propoxyphenyl)azanediyl)bis(ethan-1-ol) (3)

To the stirred mixture of salt of S1 in solution of NaOAc buffer and ACN (1:1), solution of compound S2 (R4=H, R3=OCH₂CH₂CH₃) in ACN was added slowly at 0° C. After the addition, reaction mixture was stirred for 30 mins 0° C. The completion of the reaction was monitored by TLC. The reaction mixture was then filtered and washed with ACN and water (1:1) to get the product 2,2′-((4-((2,5-dimethoxy-4-((4-nitrophenyl)diazenyl)phenyl)diazenyl)-3-propoxyphenyl) azanediyl)bis(ethan-1-ol) as purple solid. M.P.=202-203° C., MS (ESI) m/z 553 [M+H]⁺, 1H NMR (400 MHz, DMSO-d6) δ 8.41 (d, J=8.6 Hz, 2H), 8.11-7.90 (m, 2H), 7.62 (s, 1H), 7.45-7.19 (m, 2H), 6.74-6.31 (m, 2H), 4.41-4.11 (m, 2H), 4.10-3.59 (m, 16H), 1.92-1.80 (m, 2H), 1.19-1.02 (m, 3H).

### Example-4

### 2,2′-((3-butoxy-4-((2,5-dimethoxy-4-((4-nitrophenyl)diazenyl)phenyl)diazenyl)-phenyl)azanediyl)bis(ethan-1-ol) (4)

To the stirred mixture of salt of S1 in solution of NaOAc buffer and ACN (1:1), solution of compound S2 (R4=H, R3=OCH₂CH₂CH₂CH₃) in ACN was added slowly at 0° C. After the addition, reaction mixture was stirred for 30 mins 0° C. The completion of the reaction was monitored by TLC. The reaction mixture was then filtered and washed with ACN and water (1:1) to get the product 2,2′-((3-butoxy-4-((2,5-dimethoxy-4-((4-nitrophenyl)diazenyl)phenyl)diazenyl)phenyl) azanediyl)bis(ethan-1-ol) as purple solid. MS (ESI) m/z 567 [M+H]⁺, 1H NMR (400 MHz, DMSO-d6) δ 8.40 (d, J=8.4 Hz, 2H), 8.10-7.90 (m, 2H), 7.61 (s, 1H), 7.79-7.22 (m, 2H), 6.77-6.22 (m, 2H), 4.12-3.64 (m, 2H), 4.12-3.64 (m, 16H), 2.01-1.75 (m, 2H), 1.67-1.46 (m, 2H), 1.12-0.88 (m, 3H).

### Example-5

### 2,2′-((4-((2,5-dimethoxy-4-((4-nitrophenyl)diazenyl)phenyl)diazenyl)-3-(hexyloxy)phenyl) azanediyl)bis(ethan-1-ol) (5)

To the stirred mixture of salt of S1 in solution of NaOAc buffer and ACN (1:1), solution of compound S2 (R4=H, R3=OCH₂CH₂CH₂CH₂CH₂CH₃) in ACN was added slowly at 0° C. After the addition, reaction mixture was stirred for 30 mins 0° C. The completion of the reaction was monitored by TLC. The reaction mixture was then filtered and washed with ACN and water (1:1) to get the product 2,2′-((4-((2,5-dimethoxy-4-((4-nitrophenyl)diazenyl)phenyl)diazenyl)-3-(hexyloxy)phenyl)azanediyl)bis(ethan-1-ol) as purple solid. M.P.=198-199° C., MS (ESI) m/z 595 [M+H]⁺, 1H NMR (400 MHz, DMSO-d6) δ 8.41 (d, J=8.5 Hz, 2H), 8.11-7.92 (m, 2H), 7.68-7.53 (m, 1H), 7.45-7.21 (m, 2H), 6.75-6.30 (m, 2H), 4.47-4.11 (m, 2H), 4.08-3.60 (m, 16H), 1.99-1.76 (m, 2H), 1.63-1.44 (m, 2H), 1.40-1.27 (m, 4H), 0.87 (t, J=6.5 Hz, 3H).

### Example-6

### 2,2′-((4-((2,5-dimethoxy-4-((4-nitrophenyl)diazenyl)phenyl)diazenyl)-3-methylphenyl)azanediyl)bis(ethan-1-ol) (6)

To the stirred mixture of salt of S1 in solution of NaOAc buffer and ACN (1:1), solution of compound S2 (R4=H, R3=CH₃) in ACN was added slowly at 0° C. After the addition, reaction mixture was stirred for 30 mins 0° C. The completion of the reaction was monitored by TLC. The reaction mixture was then filtered and washed with ACN and water (1:1) to get the product 2,2′-((4-((2,5-dimethoxy-4-((4-nitrophenyl)diazenyl)phenyl)diazenyl)-3-methylphenyl)azanediyl)-bis(ethan-1-ol) as purple solid. M.P.=201-202° C., MS (ESI) m/z 509 [M+H]⁺, 1H NMR (400 MHz, DMSO-d6) δ 8.48-8.39 (m, 2H), 8.10-8.01 (m, 2H), 7.64 (d, J=9.9 Hz, 1H), 7.44 (s, 1H), 7.37 (s, 1H), 6.75-6.69 (m, 2H), 4.86 (t, J=5.2 Hz, 2H), 4.00 (s, 3H), 3.95 (s, 3H), 3.65-3.55 (m, 8H), 2.67 (s, 3H). 13C NMR (101 MHz, DMSO) δ 156.22, 153.67, 152.25, 150.76, 148.49, 147.24, 142.54, 142.25, 141.54, 125.57, 123.91, 118.02, 112.68, 110.76, 101.35, 100.35, 58.72, 56.84, 56.80, 53.73, 18.48.

### Example-7

### 2,2′-((4-((2,5-dimethoxy-4-((4-nitrophenyl)diazenyl)phenyl)diazenyl)-2,5-dimethoxyphenyl) azanediyl)bis(ethan-1-ol)(7)

To the stirred mixture of salt of S1 in solution of NaOAc buffer and ACN (1:1), solution of compound S2 (R₃=OCH₃ R₄=OCH₃) in ACN was added slowly at 0° C. After the addition, reaction mixture was stirred for 30 mins 0° C. The completion of the reaction was monitored by TLC. The reaction mixture was then filtered and washed with ACN and water (1:1) to get the product 2,2′-((4-((2,5-dimethoxy-4-((4-nitrophenyl)diazenyl)phenyl)diazenyl)-2,5-dimethoxyphenyl) azanediyl)bis(ethan-1-ol) as purple solid. M.P.=230-231° C., MS (ESI) m/z 555 [M+H]⁺, 1H NMR (400 MHz, DMSO-d6) δ 8.32 (d, J=8.8 Hz, 2H), 8.00 (d, J=8.8 Hz, 2H), 7.78 (d, J=9.2 Hz, 1H), 7.53 (s, 1H), 7.4 (s, 1H), 7.0 (d, J=2.34 Hz, 1H), 6.66 (dd, J=2.4, 9.2 Hz, 1H), 4.03 (s, 3H), 4.0 (s, 3H), 3.85 (s, 3H), 3.75 (s, 3H), 3.52 (t, J=4.95 Hz, 4H), 3.23 (t, J=4.96 Hz, 4H).

### Example-8

### 2,2′-((3-bromo-4-((2,5-dimethoxy-4-((4-nitrophenyl)diazenyl)phenyl)diazenyl)phenyl) azanediyl)bis(ethan-1-ol) (8)

To the stirred mixture of salt of S1 in solution of NaOAc buffer and ACN (1:1), solution of compound S2 (R4=H, R3=Br) in ACN was added slowly at 0° C. After the addition, reaction mixture was stirred for 30 mins at 0° C. The completion of the reaction was monitored by TLC. The reaction mixture was then filtered and washed with ACN and water (1:1) to get the product 2,2′-((3-bromo-4-((2,5-dimethoxy-4-((4-nitrophenyl)diazenyl)-phenyl)diazenyl)phenyl) azanediyl)bis(ethan-1-ol) as purple solid. M.P.=220-221° C., MS (ESI) m/z 573 [M+H]⁺, 1H NMR (400 MHz, DMSO-d6) δ 8.32 (d, J=8.8 Hz, 2H), 8.00 (d, J=8.8 Hz, 2H), 7.78 (d, J=9.2 Hz, 1H), 7.53 (s, 1H), 7.4 (s, 1H), 7.0 (d, J=2.34 Hz, 1H), 6.66 (dd, J=2.4, 9.2 Hz, 1H), 4.03 (s, 3H), 4.0 (s, 3H), 3.82 (t, J=4.95 Hz, 4H), 3.63 (t, J=4.96 Hz, 4H).

### Example-9

### 2,2′-((4-((4-((2,6-dichloro-4-nitrophenyl)diazenyl)-2,5-dimethoxyphenyl)diazenyl)-3-methoxyphenyl)azanediyl)bis(ethan-1-ol) (9)

To the stirred mixture of salt of S1 in solution of NaOAc buffer and ACN (1:1), solution of compound S2 (R3=OCH₃, R4=H) in ACN was added slowly at 0° C. After the addition, reaction mixture was stirred for 30 mins at 0° C. The completion of the reaction was monitored by TLC. The reaction mixture was then filtered and washed with ACN and water (1:1) to get the 2,2′-((4-((4-((2,6-dichloro-4-nitrophenyl)diazenyl)-2,5-dimethoxyphenyl)diazenyl)-3-methoxyphenyl)azanediyl)bis(ethan-1-ol) as purple solid. M.P.=189-190° C., MS (ESI) m/z 592 [M+H]⁺, 1H NMR (400 MHz, DMSO-d6) δ 8.49 (s, 2H), 7.65 (d, J=9.38 Hz, 1H), 7.37 (s, 1H), 7.31 (s, 1H), 6.53 (d, J=7.61 Hz, 1H), 6.40 (s, 1H), 4.0-3.94 (m, 6H), 3.92 (s, 3H), 3.69-3.59 (m, 8H).

### Example-10

### 2,2′-((4-((2,5-dimethoxy-4-((4-nitrophenyl)diazenyl)phenyl)diazenyl)-3-hydroxyphenyl)azanediyl)bis(ethan-1-ol) (10)

To the stirred mixture of salt of S1 in solution of NaOAc buffer and ACN (1:1), solution of compound S2 (R3=OH, R4=H) in ACN was added slowly at 0° C. After the addition, reaction mixture was stirred for 30 mins at 0° C. The completion of the reaction was monitored by TLC. The reaction mixture was then filtered and washed with ACN and water (1:1) to get the product 2,2′-((4-((2,5-dimethoxy-4-((4-nitrophenyl)diazenyl)phenyl)diazenyl)-3-hydroxyphenyl)azanediyl)-bis(ethan-1-ol as purple solid. M.P.=203-204° C., MS (ESI) m/z 551[M+H]⁺, 1H NMR (400 MHz, DMSO-d6) δ 8.47-8.40 (m, 2H), 8.09-8.03 (m, 2H), 7.62 (d, J=9.3 Hz, 1H), 7.44 (s, 1H), 7.28 (s, 1H), 6.48 (dd, J=9.4, 2.5 Hz, 1H), 6.40 (d, J=2.5 Hz, 1H), 5.03-4.80 (m, 2H), 3.99 (s, 3H), 3.97 (s, 3H), 3.68-3.57 (m, 8H).

### Example-11

### 4-(2-((2-(bis(4-methoxyphenyl)(phenyl)methoxy)ethyl)(4-((2,5-dimethoxy-4-((4-nitrophenyl)diazenyl)phenyl)diazenyl)-3-methoxyphenyl)amino)ethoxy)-4-oxobutanoic acid. (11)

To the stirred mixture of salt of S1 in solution of NaOAc buffer and ACN (1:1), solution of compound S4 (R4=H, R3=OCH3) in ACN was added slowly at 0° C. After the addition, reaction mixture was stirred for 30 mins at 0° C. The completion of the reaction was monitored by TLC. The reaction mixture was than filtered and washed with ACN and water (1:1) to get the 4-(2-((2-(bis(4-methoxyphenyl)(phenyl)methoxy)ethyl)(4-((2,5-dimethoxy-4-((4-nitrophenyl)diazenyl)phenyl)diazenyl)-3-methoxyphenyl)amino)ethoxy)-4-oxobutanoic acid as purple solid. M.P.=208-209° C., MS (ESI) m/z 927 [M+H]⁺, 1H NMR (400 MHz, DMSO-d6) δ 12.1 (brs, 1H), 8.44 (d, J=8.8 Hz, 2H), 8.06 (d, J=8.8 Hz, 2H), 7.59-7.57 (m, 1H), 7.44 (s, 1H), 7.36-7.34 (m, 2H), 7.28-7.20 (m, 7H), 7.08 (d, J=8.8 Hz, 2H), 6.84 (d, J=9.2 Hz, 4H), 6.47-6.45 (m, 1H), 6.17-6.22-6.20 (m, 1H), 4.29-4.27 (m, 2H), 3.99-3.94 (m, 9H), 3.83-3.68 (m, 10H), 3.28-3.25 (m, 2H), 2.52-2.41 (m, 4H).

### Photophysical Studies of the Compounds of General Formula I

The photophysical properties of all the synthesized compounds 1-6 were examined by UV-vis absorption analysis. Table 1 showed absorption maxima and quenching range in PCR buffer (pH 7.2).

TABLE 1
Photophysical properties of Examples 1-9.

| Example | λ_{max, abs} (nm) | Absorption range (nm) |
|---|---|---|
| 1 | 563 | 450-700 |
| 2 | 567 | 450-700 |
| 3 | 562 | 500-700 |
| 4 | 562 | 500-700 |
| 5 | 550 | 500-700 |
| 6 | 550 | 500-700 |
| 7 | 591 | 450-650 |
| 8 | 475 | 450-600 |
| 9 | 550 | 450-700 |

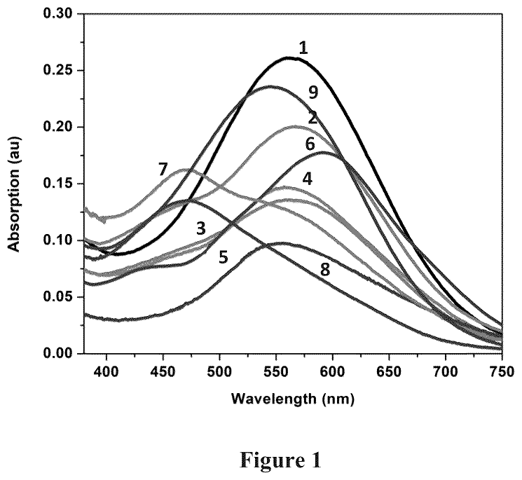

All the synthesized quenchers shown by representative examples 1-9 exhibited broad absorption spectra in PCR buffer at room temperature. The absorption spectra of newly synthesized quencher derivatives (1-9) is provided in FIG. 1. Among them compound 1 and 2 showed absorption in the range of 400-750 nm with good intensity. Similarly increasing the methylene unit of the alkoxy group in (R₄=H, R₃=OC₁-C₆) of the general formula I, a decrease in intensity was observed (FIG. 1).

### Comparison of Known Quencher BHQ-2 and the New Quencher CDRI-Q2 of the Present Invention

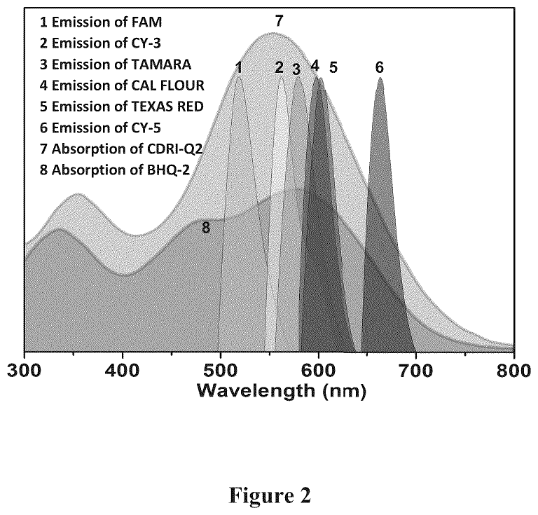

The absorption of the quencher CDRI-Q2 (Example 1) of the present invention was compared with the absorption spectrum of the known commercial quencher BHQ-2. The data suggested that the quencher CDRI-Q2 of the present invention showed broad absorption and better intensity as compared to BHQ-2 (FIG. 2).

### Quenching Studies of the CDRI-Q2 in the Presence of Different Fluorescent Dyes

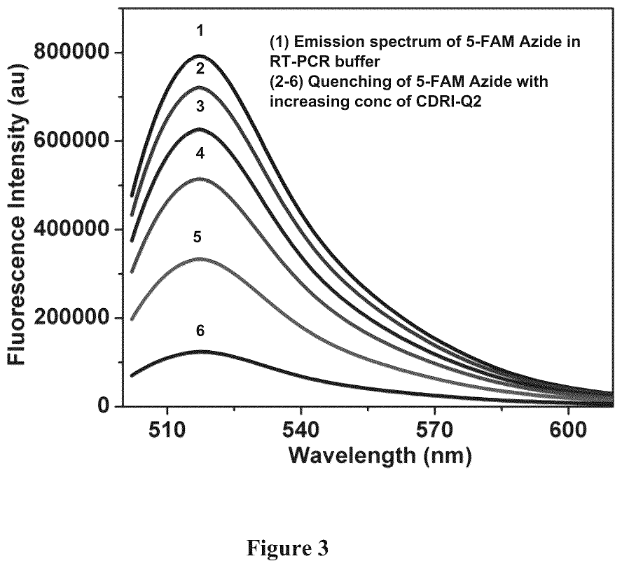

The compound CDRI-Q2 of the present invention showed effective quenching of fluorescent dye 5-FAM which showed emission maximum at 517 nm in PCR buffer solution (FIG. 3).

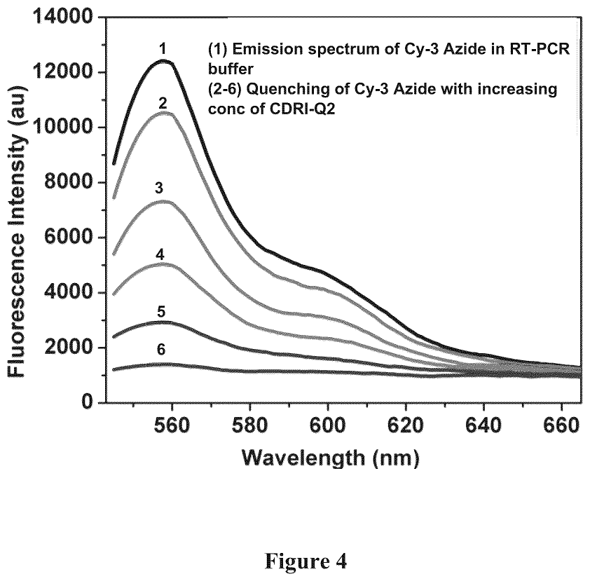

The compound CDRI-Q2 of the present invention showed effective quenching of fluorescent dye Cy-3 which showed emission maximum at 566 nm in PCR buffer solution (FIG. 4).

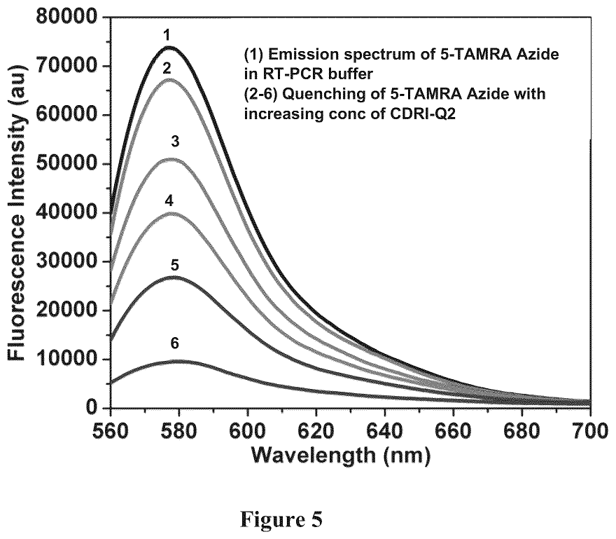

The compound CDRI-Q2 of the present invention showed effective quenching of fluorescent dye 5-TAMRA which showed emission maximum at 583 nm in PCR buffer solution (FIG. 5).

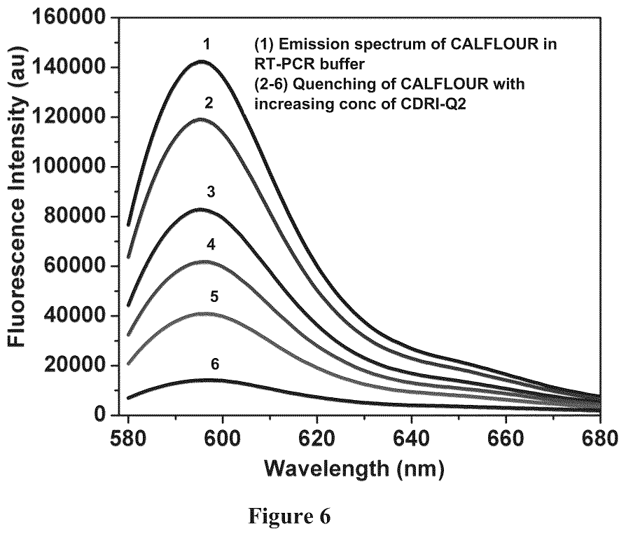

The compound CDRI-Q2 of the present invention showed effective quenching of fluorescent dye CalFluor red which showed emission maximum at 601 nm in PCR buffer solution (FIG. 6).

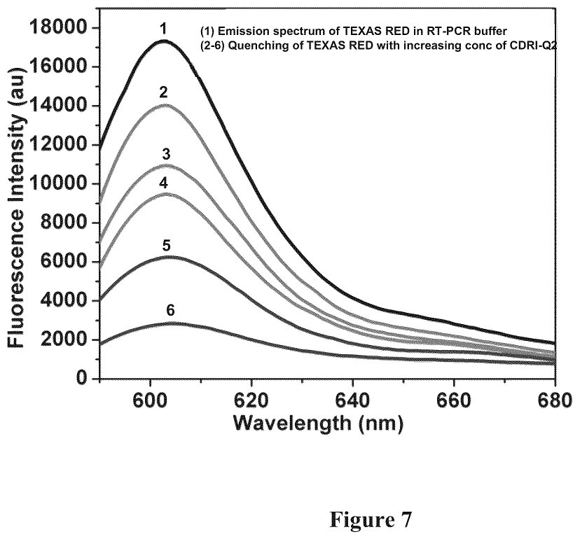

The compound CDRI-Q2 of the present invention showed effective quenching of fluorescent dye Texas Red which showed emission maximum at 603 nm in PCR buffer solution (FIG. 7).

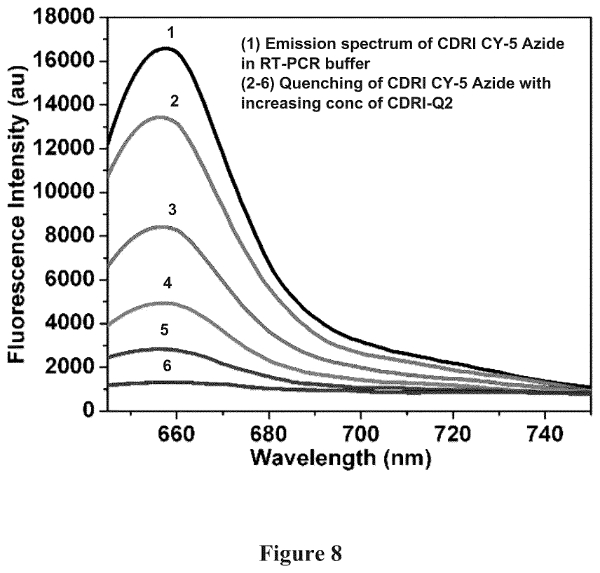

The compound CDRI-Q2 of the present invention showed effective quenching of fluorescent dye Cy-5 which showed emission maximum at 662 nm in PCR buffer solution (FIG. 8).

### Synthesis of Dual-Labelled Probe.

To a mixture of terminal acid containing fluorescence quencher of general formula I (3 eq.), DMAP (0.05 eq.), triethylamine (13 eq.), DEC/EDC (10 eq.), and anhydrous pyridine (2 mL) added free amine containing Controlled Pore Glass (CPG beads, 1000 A), then shaken at room temperature for 24 h. The solvent was removed by suction filtration and washed successively with pyridine and DCM and dried under vacuum for few hours. Then the coupling efficiency was determined by using detritylation method.

### Fluorophore Labeling at 5′-End of 3′-Quencher Tagged Oligonucleotides:

The probes for RT-PCR based diagnosis of COVID-19 were generated. CPG-amine (1000 Angs) was tagged with a novel quencher CDRI Q2 taken as representative from general formula I. Using CPG-CDRI-Q2, synthesis of oligonucleotide was performed as shown above. The oligonucleotide sequences corresponding to one host and two viral different genes E and RdRp (but not limited to these viral genes) are given in Table 2. Using phosphoramidite based solid-phase synthesis, a hexynyl group was introduced at the 5′-end of each 3′-CDRI Q2 tagged oligonucleotide, with hexynyl phosphoramidite. Fluorophore azides were obtained commercially as well as synthesized in-house.

TABLE 2
Oligonucleotide sequences for probes for RNaseP
(host) and viral E and RdRP genes

| Oligo | Sequence |
|---|---|
| RnaseP-gene | TTCTGACCTGAAGGCTCTGCGCG |
| E-gene | ACACTAGCCATCCTTACTGCGCTTCG |
| RdRp-gene | CAGGTGGAACCTCATCAGGAGATGC |

Fluorophore azides were coupled to hexynyl-oligonucleotide-3′-CDRI Q2 by Cu(I)-Catalyzed Azide-Alkyne 1,3-dipolar cycloaddition reaction, also known as Copper catalyzed alkyne azide cycloaddition (CuAAC). The copper-catalyzed reaction allows the synthesis of the 1,4-disubstituted regioisomers specifically.

### Advantages of Using Fluorophore Azides in Place of Fluorophore Phosphoramidites

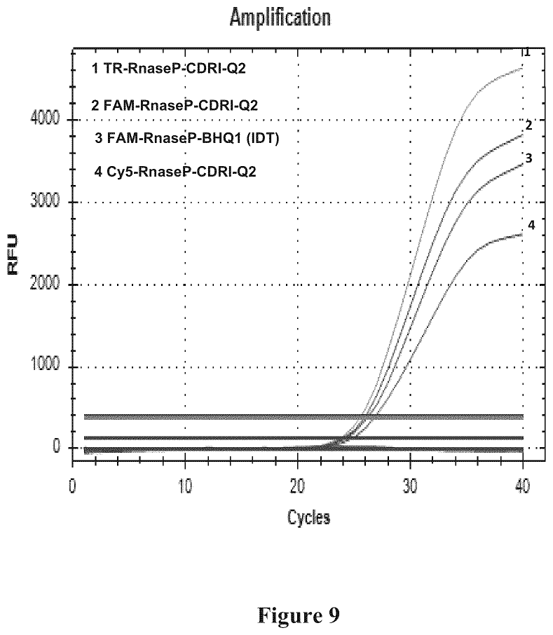

In general, dual labelled probes are prepared by attaching fluorophore at 5′-end of oligonucleotides having 3′-quencher using phosphoramidite chemistry. These fluorophore phosphoramidites are stored at −20° C. and they are not stable at room temperature and are also moisture sensitive. In the present invention, we used fluorophore azides which are stable at room temperature and are not hygroscopic in nature. Such triazole-based dual labelled oligonucleotides having different viral gene sequences (E-gene, RdRp and human gene RNaseP) are not being used for the detection of SARS-Cov2 or related viral infections using RTPCR techniques. The results of triplexing RT-PCR experiments are mentioned in FIGS. 9-11 in the drawing accompanying the specification. The details of different fluorophore azides used for labelling are given in Table 3.

TABLE 3
Photophysical of different fluorophore azides

| | λ_{abs}/λₑₘ | Molecular Weight |
|---|---|---|
| Fluorophores | (nm) | (g/mol) |
| 6-FAM-azide | 496/516 | 460.44 |
| Cyanine-5-azide | 646/662 | 565.37 |
| 5/6-Texas Red- | 584/603 | 806.95 |
| PEG₃-azide | | |

The details of the reagents and stock concentrations used for conjugate chemistry are given in Table 4.

TABLE 4
Reagents and stock concentrations used for labeling.

| | Molecular Weight | Stock |
|---|---|---|
| Reagents | (g/mol) | concentrations |
| Oligonucleotides | Variable | 500 μM |
| Triethyl amine | 101.19 | 2M |
| DMSO | 78.13 | 100% |
| Fluorophore | Variable | 10 mM |
| Ascorbic acid | 176.12 | 5 mM |
| Cupric Sulphate | 249.69 | 20.02 mM |
| Tris[(1-benzyl-1H-1,2,3- | 530.63 | 19.87 mM |
| Triazol-4-yl)methyl] amine | | |

500 μM stocks of the 5′ modified oligonucleotide were made in Nuclease Free Water (NFW) (Sigma Cat no) and 10 mM stocks of fluorescent dyes were made in molecular biology grade DMSO (Sigma).

For a 100 μL CuAAC reaction, 50 μM of alkynated oligo solution (10 μL from 500 μM stock in NFW) was sequentially treated with 0.2 M of Triethylammonium acetate buffer, pH 7.0 (Sigma) followed by the addition of 50 μL DMSO. The reaction was mixed properly by vortexing. 150 μM of the fluorescent azide solution (1.5 μL from 10 mM stock in DMSO) and 0.5 mM of freshly prepared ascorbic acid solution in NFW were further added to the reaction mixture, with proper mixing after addition of each reagent. The reaction mix was then degassed properly and flushed with argon for about 60 seconds. 0.5 mM of Copper (II)-Tris [(1-benzyl-1H-1,2,3-trizole-4-yl)methyl]amine complex (Cu-TBTA complex, prepared by mixing 5 mg/mL copper (II) sulphate pentahydrate and 10.5 mg/mL of TBTA in 55% DMSO) was added to the reaction mixture, mixed thoroughly by vortexing and was again flushed with argon for another 60-100 seconds. The reaction was incubated for 12-16 hours at 22° C. After the completion of the reaction, the reaction was precipitated by adding 3 volumes of chilled acetone and stored at −20° C. for 30 minutes. The labeled DNA was extracted by high-speed centrifugation of the mixture at 10,000 rpm for 20 minutes, at 4° C.

The pellet obtained at this step was washed twice with 1 mL of chilled acetone. The pellet obtained after final washing was dried by further incubating the tube at 22° C. for approximately 30 minutes. The dried fluorophore-labeled oligonucleotide obtained at this step was resuspended in 45 μL of chilled NFW for HPLC purification.

Analytical purification of the labeled oligonucleotide was carried out by HPLC using a dual pump Shimadzu HPLC system equipped with 20 μL sample loop, and RF-20A spectrofluorometric and SPD-10A UV-VIS detectors, over an XTerra MS C18 column (75×4.6 mm packed with 2.5 μm particles, average pore diameter 125 Å) with an Inertsil C4 5 μm guard column (4.0×10 mm). The fluorescence detector was set with the corresponding excitation and emission wavelengths for the fluorophore of interest, while 260 and 280 nm wavelengths were set in the UV detector. The mobile phase was composed of 0.1 M triethylammonium acetate buffer, pH 7.0 (Sigma), and acetonitrile (HPLC grade, Sigma). The oligos were separated by running an acetonitrile gradient of 0-60% over 30 minutes through the column, at a flow rate of 1 mL/min. The peaks corresponding to both the fluorescent and UV detection were collected manually and stored at −20° C. These stored samples were frozen in liquid nitrogen and lyophilized in CHRiST lyophilization system at 0.08 mbar and −51° C. The lyophilized probes were stored at −20° C. and used in RT-PCR for detection of the respective genes.

### Demonstration of Application of Quenchers in RT-PCR Based Diagnosis of SARS Cov-2 Infection

### Assay Procedure:

1) Extract RNA using commercially available kits. For RT-PCR reaction, a single tube RT-mix allows first-strand synthesis of cDNA from RNA molecules followed by PCR amplification and detection using specific primer-probe. Depending upon the abundance of target RNA, template concentration can be used in the range of 0.5 pg-0.5 μg. Alternatively, the reaction can be performed separately by cDNA synthesis (0.5 μg-2 μg). The cDNA can be diluted 3-5 times and used for PCR amplification and detection using specific primer-probe.
2) For reaction set-up for real time PCR and cycling protocol, follow manufacture's protocol. The primers (Forward and Reverse) and probe concentration can be used 0.2 μM-1 μM.
3) The fluorophore-quencher is compatible for detection of target genes in various real-time PCR instruments (ABI, BioRad) using Fluorophore specific channels.

FIG. 9 provides RT-PCR data representation with the cycle threshold (Ct) on X-axis and RFU on Y-axis. FAM-RnaseP-BHQ1 (from IDT) was used as a positive control. Probes used for detection had CDRI-Q2 at 3′end and FAM/Texas red (TR)/Cy5 at 5′end. Data demonstrates the CDRI-Q2 quenching compatibility in diverse range (520 nm-670 nm) for accurate RT-PCR based detection.

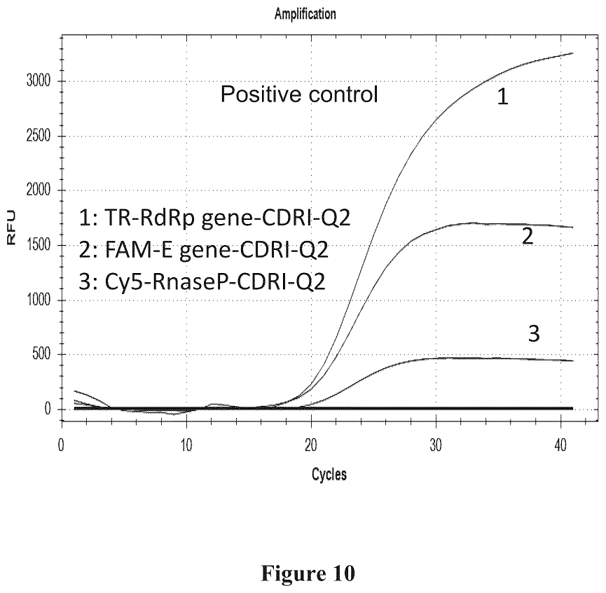

Multiplexing RT-PCR based detection of SARS-CoV-2 viral genes E and RdRp and RnaseP as housekeeping gene was conducted using positive Control; data represents with the cycle threshold (Ct) on X-axis and RFU on Y-axis as shown in FIG. 10. Data demonstrated the CDRI-Q2 quenching compatibility in diverse emission range (450 nm-700 nm) for RT-PCR.

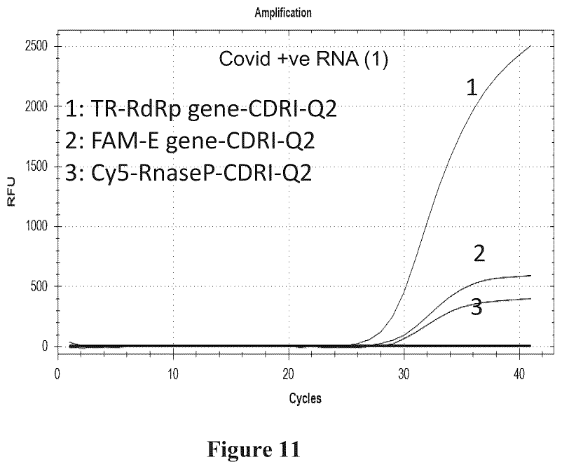

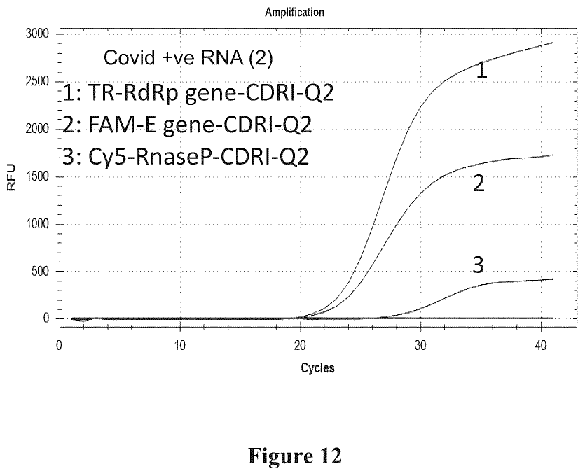

Also, FIG. 11 illustrates Multiplexing RT-PCR based detection of SARS-CoV-2 viral genes E and RdRp and Rnase P as housekeeping gene using positive RNA samples from COVID-19 positive patients; data represents with the cycle threshold (Ct) on X-axis and RFU on Y-axis. FAM-E-CDRI-Q2, TR-RdRp-CDRI-Q2 and Cy5-RnaseP-CDRI-Q2 probes were used. Data demonstrates the CDRI-Q2 quenching compatibility in diverse emission range (450 nm-700 nm) for RT-PCR.

### Advantages of the Present Invention

The present invention provides a family of significantly non-fluorescent quenchers of excited state energy, well-defined modified quenchers of already known BHQ-2 (“Black Hole Quencher”). As per the literature, BHQ-2 is good for the dyes that emit in the orange-red part of visible range (560-670 nm), and it is not suitable for FAM. For FAM, BHQ-1 is preferably used. The present invention provides a class of universal quenchers that are functionalized to allow their rapid attachment to probe components and provides quenchers that are engineered to have a desired broad quenching range covering of entire visible spectrum. The present invention illustrate use of fluorophore azides which are stable at room temperature and are not hygroscopic in nature

A quencher may consist of electron donating and withdrawing groups combining together by a pi-conjugating network. By modifying conjugated system of quencher and/or incorporating electron donating and withdrawing groups onto aromatic scaffold, the spectral properties (e.g., absorbance) can be “tuned” to match the spectral characteristics (e.g., emission) of one or more fluorophores. New quenchers of the present invention showed broad absorption spectra covering entire visible color range. These quenchers can be used to quench different fluorophores which emit in the range between 500-750 nm such as FAM, Cyanine dyes, Texas red, Calfluor red, as well as other fluorescent dyes. Moreover, it has better quenching properties such as higher absorbance than other well-known BHQ-dyes.

1. A compound of general formula I, nucleic acid conjugates, complexes and salts thereof 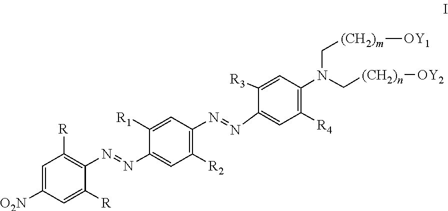
wherein
R is independently selected from the group consisting of hydrogen and halogen;
R₁ and R₂ are independently selected from the group consisting of hydrogen, substituted or unsubstituted (C₁-C₄) alkyl and (C₁-C₆) alkoxy;
R₃ is selected from the group consisting of hydroxy, halogen, (C₁-C₆) alkoxy, substituted or unsubstituted (C₁-C₄) alkyl, thioalkyl (SC₁-C₆), methylamino and dimethylamino;
R₄ is selected from the group consisting of hydrogen, hydroxy, halogen, (C₁-C₆) alkoxy, and substituted or unsubstituted (C₁-C₄) alkyl;
m and n are independently selected from 0 to 3;
Y₁ and Y₂ are independently selected from the group consisting of hydrogen, (C₁-C₆) alkyl, glycol, substituted or unsubstituted alkylaryl, oxo-alkanoic acid, epoxy, N-hydroxysuccinimide ester, N-hydroxybenztriazole ester, acid halide, acyl imidazole, thioester, p-nitrophenyl ester, alkyl ester, phosphoramidite, mononucleotide unit, two or more mononucleotide units with or without separate phosphate or polyphosphate groups linked by nucleoside groups.

2. The compounds claimed in claim 1, wherein the compound is selected from the group consisting of:
i. 2,2′-((4-((2,5-dimethoxy-4-((4-nitrophenyl)diazenyl)phenyl)diazenyl)-3-methoxyphenyl)azanediyl)bis(ethan-1-ol) (1),
ii. 2,2′-((4-((2,5-dimethoxy-4-((4-nitrophenyl)diazenyl)phenyl)diazenyl)-3-ethoxyphenyl)azanediyl)bis(ethan-1-ol) (2),
iii. 2,2′-((4-((2,5-dimethoxy-4-((4-nitrophenyl)diazenyl)phenyl)diazenyl)-3-propoxyphenyl)azanediyl)bis(ethan-1-ol) (3),
iv. 2,2′-((3-butoxy-4-((2,5-dimethoxy-4-((4-nitrophenyl)diazenyl)phenyl)diazenyl)phenyl)azanediyl)bis(ethan-1-ol) (4),
v. 2,2′-((4-((2,5-dimethoxy-4-((4-nitrophenyl)diazenyl)phenyl)diazenyl)-3-(hexyloxy)phenyl)azanediyl)bis(ethan-1-ol) (5),
vi. 2,2′-((4-((2,5-dimethoxy-4-((4-nitrophenyl)diazenyl)phenyl)diazenyl)-3-methylphenyl)azanediyl)bis(ethan-1-ol) (6),
vii. 2,2′-((4-((2,5-dimethoxy-4-((4-nitrophenyl)diazenyl)phenyl)diazenyl)-2,5-dimethoxyphenyl)azanediyl)bis(ethan-1-ol)(7),
viii. 2,2′-((3-bromo-4-((2,5-dimethoxy-4-((4-nitrophenyl)diazenyl)phenyl)diazenyl)phenyl) azanediyl)bis(ethan-1-ol)(8),
ix. 2,2-((4-((4-((2,6-dichloro-4-nitrophenyl)diazenyl)-2,5-dimethoxyphenyl)diazenyl)-3-methoxyphenyl)azanediyl)bis(ethan-1-ol) (9),
x. 22′-((4-((2,5-dimethoxy-4-((4-nitrophenyl)diazenyl)phenyl)diazenyl)-3-hydroxyphenyl)azanediyl)bis(ethan-1-ol) (10),
xi. 4-(2-((2-(bis(4-methoxyphenyl)(phenyl)methoxy)ethyl)(4-((2,5-dimethoxy-4-((4-nitrophenyl)diazenyl)phenyl)diazenyl)-3-methoxyphenyl)amino)ethoxy)-4-oxobutanoic acid (11),
xii. 4-(2-((2-(bis(4-methoxyphenyl)(phenyl)methoxy)ethyl)(4-(2,5-dimethoxy-4-((4-nitrophenyl)diazenyl)phenyl)diazenyl)-3-ethoxyphenyl)amino)ethoxy)-4-oxobutanoic acid (12),
xiii. 4-(2-((2-(bis(4-methoxyphenyl)(phenyl)methoxy)ethyl)(4-((2,5-dimethoxy-4-((4-nitrophenyl)diazenyl)phenyl)diazenyl)-3-propoxyphenyl)amino)ethoxy)-4-oxobutanoic acid (13),
xiv. 4-(2-((2-(bis(4-methoxyphenyl)(phenyl)methoxy)ethyl)(3-butoxy-4-((2,5-dimethoxy-4-((4-nitrophenyl)diazenyl)phenyl)diazenyl)phenyl)amino)-ethoxy)-4-oxobutanoic acid (14),
xv. 4-(2-((2-(bis(4-methoxyphenyl)(phenyl)methoxy)ethyl)(4-((2,5-dimethoxy-4-((4-nitrophenyl)diazenyl)phenyl)diazenyl)-3-(hexyloxy)phenyl)amino)-ethoxy)-4-oxobutanoic acid, (15) and
xvi. 4-(2-((2-(bis(4-methoxyphenyl)(phenyl)methoxy)ethyl)(4-((2,5-dimethoxy-4-((4-nitrophenyl)diazenyl)phenyl)diazenyl)-3-methylphenyl)amino)-ethoxy)-4-oxobutanoic acid (16).

3. The compound as claimed in claim 1, wherein its nucleic acid conjugate comprises nucleotide sequence selected from the group consisting of SEQ ID NO: 1-3.

4. A process for the preparation of compound of general formula I, nucleic acid conjugates, complexes, and salts thereof, 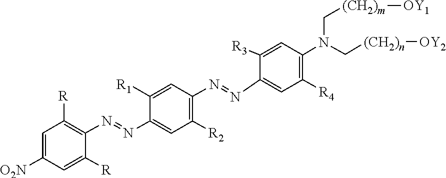
wherein R is selected from the group consisting of hydrogen and halogen;
R₁ and R₂ are independently selected from the group consisting of hydrogen, substituted or unsubstituted (C₁-C₄) alkyl and (C₁-C₆) alkoxy;
R₃ is selected from the group consisting of hydroxy, halogen, (C₁-C₆) alkoxy, substituted or unsubstituted (C₁-C₄) alkyl, thioalkyl (SC₁-C₆), methylamino and dimethylamino;
R₄ is selected from the group consisting of hydrogen, hydroxy, halogen, (C₁-C₆) alkoxy, and substituted or unsubstituted (C₁-C₄) alkyl;
m and n are independently selected from 0 to 3;
Y₁ and Y₂ are independently selected from the group consisting of hydrogen, (C₁-C₆) alkyl, glycol, substituted or unsubstituted alkylaryl, oxo-alkanoic acid, epoxy, N-hydroxysuccinimide ester, N-hydroxybenztriazole ester, acid halide, acyl imidazole, thioester, p-nitrophenyl ester, alkyl ester, alkenyl ester, alkynyl ester, aromatic ester, phosphoramidite, mononucleotide unit, two or more mononucleotide units with or without separate phosphate or polyphosphate groups linked by nucleoside groups, comprising the steps of: 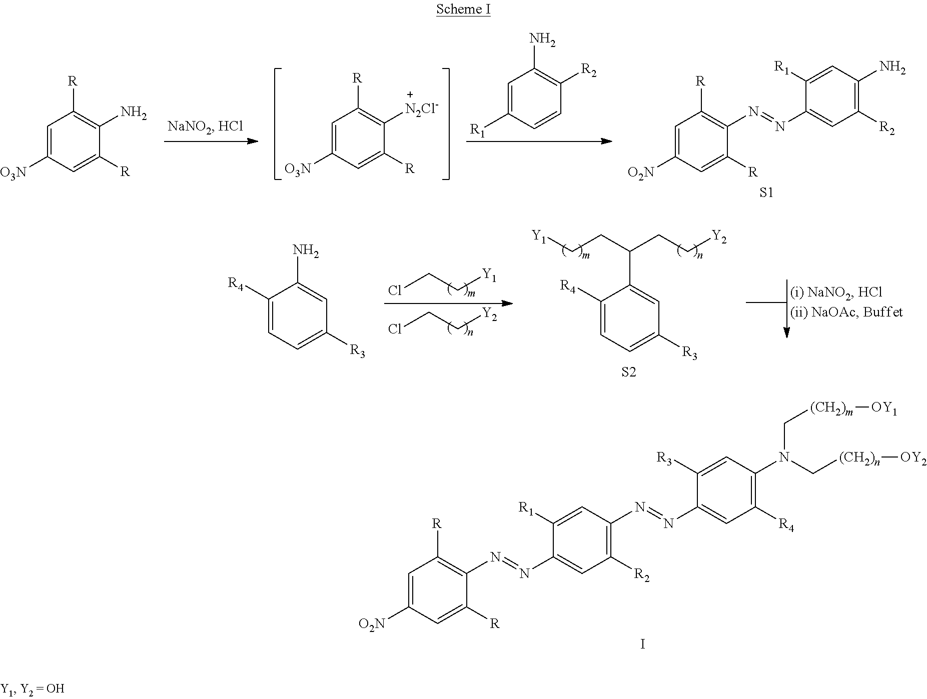
a. reacting a solution of unsubstituted or substituted 4-nitroaniline in HCl with a solution of sodium nitrite in distilled water to form diazonium salts followed by reaction with substituted aniline to form a compound having general formula S1;
b. reacting a mixture of substituted aniline with substituted alkylhalide in the presence of base to form a compound having general formula S2;
c. reacting a compound having general formula S1 in HCl with a solution of sodium nitrite to form a diazonium salt, which was then reacted with a compound having general formula S2 in the presence of NaOAc buffer to obtain a compound having general formula I and
d. isolating the compound of general formula I from the reaction mixture and purifying by washing with organic solvents or by chromatography.

5. The process as claimed in claim 4, wherein steps a-c are carried out in the presence of an organic solvent selected from CH₃CN, Dimethylsulphoxide, water and tetrahydrofuran at a temperature ranging between 0° C. to 100° C. for a period ranging between 1 minute to 3 days.

6. A process for the preparation of compound of general formula I, nucleic acid conjugates, complexes, and salts thereof as claimed in claim 4, wherein the process comprises the steps of: 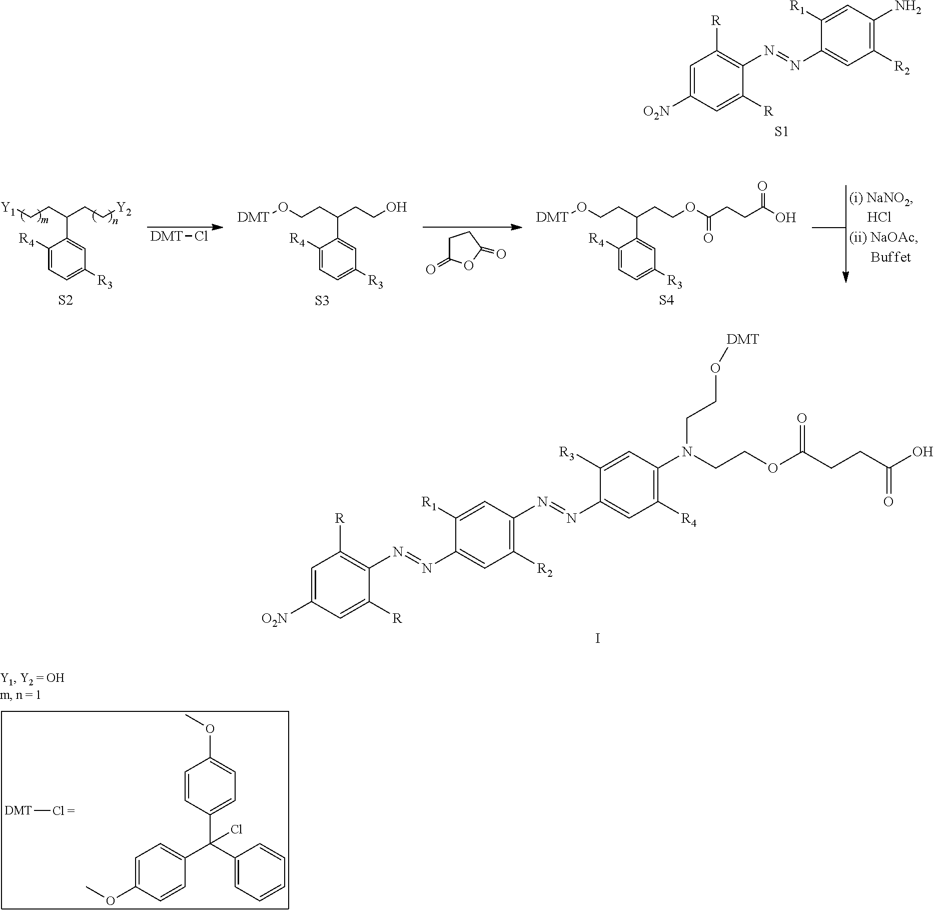
a. reacting compound S2, wherein Y₁, Y₂=OH and m, n=1 in dry DCM in the presence of a base (DIPEA) with DMT-Cl at room temperature under inert atmosphere to afford compound S3;
b. reacting S3 with succinic anhydride, and DMAP in organic solvent for 12-48 h to afford compound S4 and
c. reacting the diazonium salt of S1 with S4 to obtain the compound having general formula I.

7. A process for the preparation of conjugate compound of general formula I wherein Q is the quencher compound of formula 1 comprising the steps of: 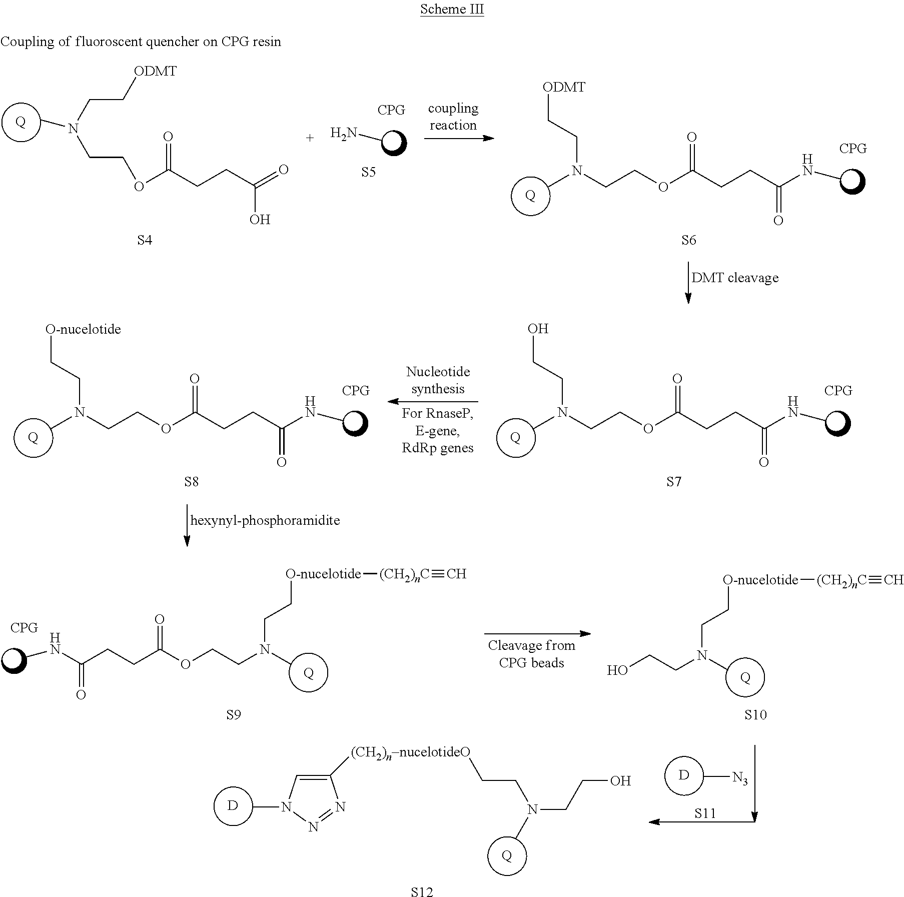
a. coupling of a compound of formula S4 with the amine functionality of the solid support CPG beads of formula S5 to produce S6 followed by deprotection of DMT group to obtain S7;
b. reacting S7 with nucleotide phosphoramidites to form oligonucleotide S8 followed by 5′-modiciation with hexynyl-phosphoramidite to afford product S9 and
c. treating S9 with abase for cleaving the oligonucleotide from the solid support to obtain S10 and
d. reacting S10 with fluorescent dye azides to furnish oligonucleotides probes of general formula S12.

8. The compound as claimed in claim 1, wherein the compound is used in combination with acetyl, azides, oxo-alkanoic acid, epoxy, N-hydroxysuccinimide ester, N-hydroxybenztriazole ester, acid halide, acyl imidazole, thioester, p-nitrophenyl ester, alkyl ester, phosphoramidite, mononucleotide unit, two or more mononucleotide units with or without separate phosphate or polyphosphate groups linked by nucleoside groups.

9. A process for detecting nucleic acids (DNA, RNA), peptides, chemicals, pharmaceuticals, microorganisms and other biological substances of diagnostic importance using the compounds as claimed in claim 1.

10. A process for detection of substances, hormones, pathogenic microorganisms, viruses, antibodies, enzymes and nucleic acids using the compounds as claimed in claim 1.

11. The compound as claimed in claim 1, wherein the compound is useful for preparing mono-, or dual labelled probe and analyzing them in single, duplexing and multiplexing in RTPCR or other related detecting systems.


SEQUENCE LISTING (3 sequences)

SEQ ID NO: 1 - DNA, 23 bp, synthetic construct
  source 1..23: mol_type = other DNA; note = RNaseP - gene
ttctgacctgaaggctctgcgcg

SEQ ID NO: 2 - DNA, 26 bp, synthetic construct
  source 1..26: mol_type = other DNA; note = E-gene
acactagccatccttactgcgcttcg

SEQ ID NO: 3 - DNA, 25 bp, synthetic construct
  source 1..25: mol_type = other DNA; note = RdRp - gene
caggtggaacctcatcaggagatgc